(19) United States
(12) Patent Application Publication
(10) Pub. No.: US 2025/0057518 A1
(43) Pub. Date: Feb. 20, 2025

(54) ADAPTER ASSEMBLIES AND SURGICAL LOADING UNITS

(71) Applicant: Covidien LP, Mansfield, MA (US)

(72) Inventors: Andriy Buyda, East Haven, CT (US); David M. Chowaniec, Rocky Hill, CT (US); Asa G. DeBlois, New Haven, CT (US)

(73) Assignee: Covidien LP, Mansfield, MA (US)

(21) Appl. No.: 18/815,468

(22) Filed: Aug. 26, 2024

Related U.S. Application Data
(63) Continuation of application No. 17/121,834, filed on Dec. 15, 2020, now Pat. No. 12,102,305.
(60) Provisional application No. 62/961,245, filed on Jan. 15, 2020.

(51) Int. Cl. A61B 17/00; A61B 17/072

(52) U.S. Cl. CPC A61B 17/00; A61B 2017/0046; A61B 2017/00482; A61B 2017/00486; A61B 2017/07285

(57) ABSTRACT

An adapter assembly includes an elongate body configured to receive a surgical loading unit and an elongate loading bar coupled to the elongate body and configured to selectively lock the surgical loading unit to the adapter assembly. The elongate loading bar has a distal end defining a slot therein configured for receipt of an articulation link of the surgical loading unit upon an improper insertion of the surgical loading unit into the adapter assembly. With the articulation captured in the slot, rotation of the surgical loading unit toward a fully assembled state with the adapter assembly is resisted.

### CROSS-REFERENCE TO RELATED APPLICATIONS

This application is a continuation of U.S. patent application Ser. No. 17/121,834 filed Dec. 15, 2020, which claims the benefit of and priority to U.S. Provisional Patent Application Ser. No. 62/961,245 filed Jan. 15, 2020, the entire disclosures of which are incorporated by reference herein. To the extent appropriate a claim of priority is made to each of the above disclosed applications.

### FIELD

The present technology is generally related to adapter assemblies for use with an electromechanical surgical system and their methods of use. More specifically, the present technology relates to electromechanical surgical instruments configured to ensure the proper connection of a loading unit with an adapter assembly.

### BACKGROUND

Linear clamping, cutting, and stapling surgical devices may be employed in surgical procedures to resect tissue. Conventional linear clamping, cutting, and stapling devices include a handle assembly, an adapter assembly extending from the handle assembly, and a surgical loading unit detachably coupled to the adapter assembly. The surgical loading unit includes a pair of opposing gripping jaw members, which clamp about the tissue. One or both of the jaw members, such as the anvil portion, moves or pivots relative to the overall structure. Actuation of the device may be controlled by a grip trigger maintained in the handle assembly or via a robotic assembly.

In addition to the jaw members, the surgical loading unit may also include a stapling mechanism. One of the jaw members of the surgical loading unit may include a staple cartridge receiving region and a mechanism for driving staples up through tissue clamped against the anvil portion, thereby sealing the tissue. The jaw members may be integrally formed with the adapter assembly or may be detachable such that various gripping and stapling elements may be interchangeable.

### SUMMARY

The techniques of this disclosure generally relate to adapter assemblies for interconnecting handle assemblies and surgical loading units. The present disclosure also relates to mechanical features that ensure a proper assembly of the surgical loading unit with the adapter assembly.

According to an aspect of the present disclosure, a surgical instrument is provided and includes a surgical loading unit and an adapter assembly. The surgical loading unit has a proximal body portion, a pair of surface features extending outwardly from the proximal body portion, an articulation link slidably coupled to the proximal body portion, and a tool assembly coupled to a distal end portion of the proximal body portion. The articulation link has a distal end portion coupled to the tool assembly and configured to articulate the tool assembly relative to the proximal body portion. The adapter assembly includes an elongate body and an elongate loading bar coupled to the elongate body. The elongate body has a distal end portion configured to receive the proximal end portion of the proximal body portion of the surgical loading unit. The elongate loading bar is configured to move relative to the elongate body between a proximal position and a distal position and has a distal end defining a slot therein. The slot is configured for receipt of a proximal end portion of the articulation link of the surgical loading unit upon an improper insertion of the surgical loading unit into the adapter assembly, whereby the engagement of the proximal end portion of the articulation link with the slot of the elongate loading bar resists rotation of the surgical loading unit relative to the adapter assembly toward an assembled state.

In aspects, the distal end portion of the elongate body may have an inner surface defining an opening, a pair of diametrically opposed apertures, and a slot. The opening may be configured for receipt of the proximal end portion of the proximal body portion of the surgical loading unit. The pair of diametrically opposed apertures may be configured for receipt of the pair of surface features of the surgical loading unit. The slot may be configured for receipt of the articulation link of the surgical loading unit during a proper insertion of the surgical loading unit into the adapter assembly.

In aspects, the elongate loading bar may have a distal extension configured to selectively lock the surgical loading unit to the adapter assembly when the elongate loading bar is in the distal position.

In aspects, a first surface feature of the pair of surface features may be configured to engage the distal extension of the elongate loading bar to move the elongate loading bar towards the proximal position during a proper insertion of the surgical loading unit into the adapter assembly.

In aspects, the distal extension of the elongate loading bar may be configured for locking engagement with the first surface feature of the surgical loading unit upon the proper insertion and rotation of the surgical loading unit into the elongate body of the adapter assembly.

In aspects, the adapter assembly may further include an annular member rotatably disposed within the distal end portion of the elongate body. The annular member may include a first surface feature configured to interface with a second surface feature of the pair of surface features of the surgical loading unit, such that the annular member is rotatable by the surgical loading unit when the second surface feature of the surgical loading unit is engaged with the first surface feature of the annular member.

In aspects, the annular member may include a second surface feature engaged with the distal extension of the elongate loading bar when the elongate loading bar is in the distal position, such that the distal extension of the elongate loading bar resists rotation of the annular member when the elongate loading bar is in the distal position.

In accordance with another aspect, an adapter assembly is provided and includes an elongate body and an elongate loading bar coupled to the elongate body. The elongate body includes a distal end portion configured to receive a surgical loading unit. The elongate loading bar is configured to move relative to the elongate body between a proximal position, in which the surgical loading unit is removable from the elongate body, and a distal position, in which the elongate loading bar locks the surgical loading unit to the elongate body. The elongate loading bar has a distal end defining a slot therein configured for receipt of an articulation link of the surgical loading unit upon an improper insertion of the surgical loading unit into the adapter assembly, whereby the engagement of the articulation link with the slot of the elongate loading bar resists rotation of the surgical loading unit relative to the adapter assembly toward an assembled state.

In aspects, the distal end portion of the elongate body may have an inner surface defining an opening, a pair of apertures in communication with the opening, and a slot in communication with the opening. The opening may be configured for receipt of the surgical loading unit. The apertures may be configured for receipt of a pair of surface features of the surgical loading unit. The slot may be configured for receipt of the articulation link of the surgical loading unit during a proper insertion of the surgical loading unit into the adapter assembly.

In aspects, the slot of the elongate body may be disposed between the pair of apertures.

In aspects, the slot of the distal end of the elongate loading bar may have substantially the same width as the slot of the elongate body.

In aspects, the elongate loading bar may have a distal extension configured to selectively lock the surgical loading unit to the adapter assembly when the elongate loading bar is in the distal position.

In aspects, the adapter assembly may further include an annular member rotatably disposed within the distal end portion of the elongate body. The annular member may include a first surface feature defining a cavity configured to interface with a lug of the surgical loading unit, such that the annular member is rotatable by the surgical loading unit when the lug of the surgical loading unit is captured in the cavity of the annular member.

In aspects, the annular member may include a second surface feature engaged with the distal extension of the elongate loading bar when the elongate loading bar is in the distal position, such that the distal extension of the elongate loading bar resists rotation of the annular member when the elongate loading bar is in the distal position.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Aspects of the present disclosure are described herein with reference to the accompanying drawings, wherein:

FIG. 1 is a perspective view, with parts separated, of components of a hand-held, electromechanical surgical instrument including a handle assembly, an adapter assembly, and a surgical loading unit;

FIG. 2 is a perspective view of a proximal end portion of the surgical loading unit of FIG. 1;

FIG. 3 is a front view of a ring member of the adapter assembly of FIG. 1 which receives the proximal end portion of the surgical loading unit;

FIG. 4 is a perspective view of the proximal end portion of the surgical loading unit being properly inserted into the ring member of FIG. 3;

FIG. 5 is a side, perspective view illustrating a first improper insertion of the surgical loading unit into the adapter assembly;

FIG. 6 is a side, perspective view illustrating a first side of the surgical instrument during a second improper insertion of the surgical loading unit into the adapter assembly; and

FIG. 7 is a perspective view illustrating another side of the surgical instrument during the second improper insertion of the surgical loading unit into the adapter assembly.

### DETAILED DESCRIPTION

As used herein, the terms parallel and perpendicular are understood to include relative configurations that are substantially parallel and substantially perpendicular up to about + or −10 degrees from true parallel and true perpendicular.

Aspects of the presently disclosed surgical instruments including handle assemblies, adapter assemblies, and surgical loading units thereof, are described in detail with reference to the drawings, in which like reference numerals designate identical or corresponding elements in each of the several views. As used herein, the term “distal” refers to that portion of the surgical instrument, adapter assembly, handle assembly, loading unit, or component thereof, farther from the user, while the term “proximal” refers to that portion of the surgical instrument, adapter assembly, handle assembly, loading unit or component thereof, closer to the user.

For more details of certain aspects of the adapter assembly disclosed herein, reference may be made to U.S. Pat. No. 10,426,466, the entire contents of which are incorporated by reference herein.

Presently, if a surgical loading unit is inserted incorrectly into an adapter assembly and rotated after the incorrect insertion, an annular member or rotating ring of the adapter assembly is caused to be rotated out of a normal position. After the surgical loading unit is removed, the rotating ring remains out of the normal position. As such, a subsequent attempt at inserting a surgical loading unit into the adapter assembly is prohibited due to the rotating ring being displaced from its normal operating position.

The present disclosure provides a surgical instrument that includes a surgical loading unit and an adapter assembly that interconnects the surgical loading unit with either a handle assembly or a robotic assembly. The adapter assembly includes a plurality of mechanical features that ensure that the surgical loading unit is connected to the adapter assembly in a proper orientation to prevent the improper displacement of the rotating ring.

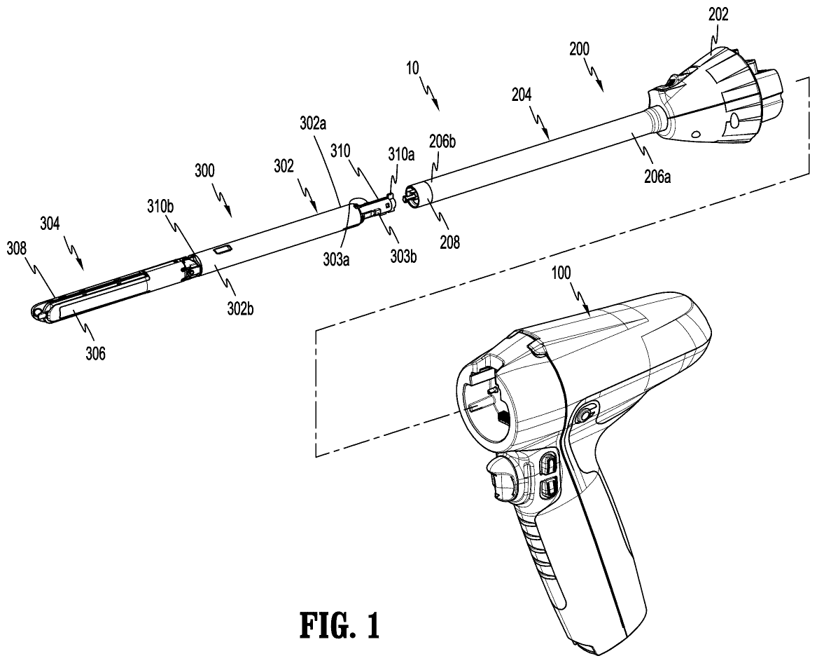

With reference to FIG. 1, a surgical instrument 10, in accordance with an aspect of the present disclosure, is shown as a powered, hand-held, electromechanical surgical instrument. The surgical instrument 10 includes a handle assembly 100 configured for selective connection with any one of a number of adapter assemblies 200, and, in turn, each unique adapter assembly 200 is configured for selective connection with any number of surgical loading units 300. The surgical loading unit 300 and adapter assembly 200 are configured for actuation and manipulation by the handle assembly 100 or, in aspects, a robotic assembly (not shown).

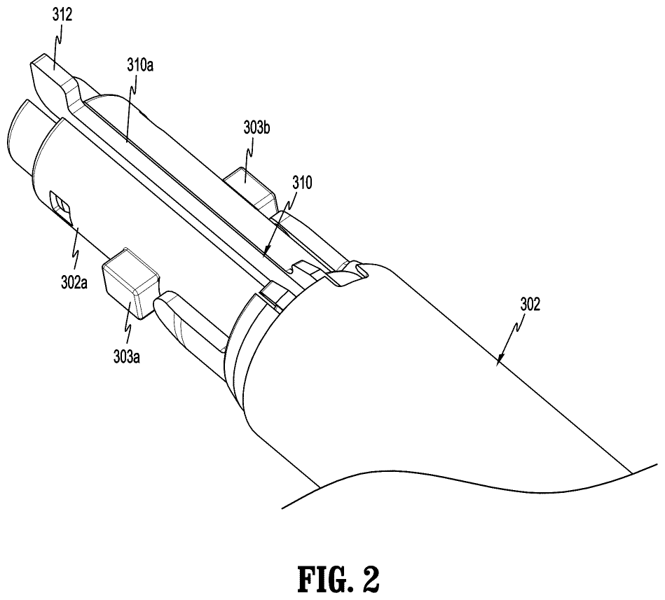

With reference to FIGS. 1 and 2, the surgical loading unit 300 of the surgical instrument 10 has a proximal body portion 302 and a tool assembly or end effector 304 coupled to a distal end portion 302b of the proximal body portion 302. The proximal body portion 302 has a proximal end portion 302a configured for engagement with a distal end portion 206b of an elongate body 204 of the adapter assembly 200. The proximal body portion 302 has a pair of surface features, such as, for example, lugs 303a, 303b extending outwardly from opposite sides of the proximal end portion 302a of the surgical loading unit 300. The lugs 303a, 303b may assume any suitable shape, such as a square or a cylinder. The end effector 304 is pivotally attached to the proximal body portion 302 and includes an anvil assembly 306 and a cartridge assembly 308. The cartridge assembly 308 is pivotable in relation to the anvil assembly 306 and is movable between an open or unclamped position and a closed or clamped position for insertion through a cannula of a trocar. In aspects, the end effector 304 may be configured to perform alternate functions, such as, electrosurgical sealing.

The surgical loading unit 300 further includes an articulation link 310 extending through the proximal body portion 302 and centrally between the lugs 303a, 303b. The articulation link 310 has a proximal end portion 310a having a flag 312 protruding proximally and radially outward from the proximal body portion 302. The flag 312 of the articulation link 310 is configured to operably couple to an articulation drive member (not explicitly shown) of the adapter assembly 200 for driving a translation of the articulation link 310. The articulation link 310 has a distal end portion 310b operably coupled to the end effector 304, such that the end effector 304 is configured to articulate relative to the proximal body portion 302 in response to a translation of the articulation link 310. For example, the end effector 304 is movable from a first position in which the end effector 304 is aligned with a longitudinal axis of the proximal body portion 302 to at least a second position in which the end effector 304 is disposed at a non-zero angle with respect to the longitudinal axis of the proximal body portion 302.

Reference may be made to U.S. Pat. No. 7,819,896, the entire contents of which are incorporated herein by reference, for a detailed discussion of the construction and operation of an exemplary end effector.

With further reference to FIG. 1, the adapter assembly 200 includes a knob housing 202 and an elongate body 204 extending from a distal end of the knob housing 202. The knob housing 202 and elongate body 204 are configured and dimensioned to house the components of the adapter assembly 200. The elongate body 204 may be dimensioned for endoscopic insertion. In aspects, the elongate body 204 may be passable through a typical trocar port, cannula or the like. The knob housing 202 may be dimensioned to not enter the trocar port, cannula of the like. The elongate body 204 has a proximal end portion 206a attached to the knob housing 202, which is configured to be attached to the handle assembly 100. The elongate body 204 also includes a distal end portion 206b configured to be coupled to the proximal body portion 302 of the surgical loading unit 300.

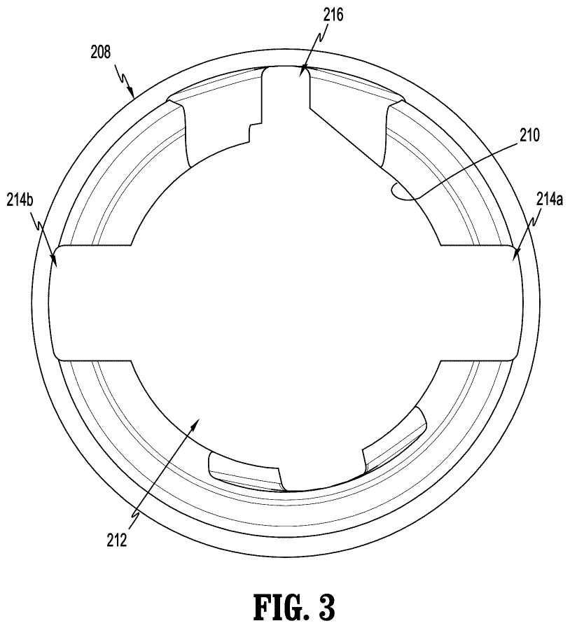

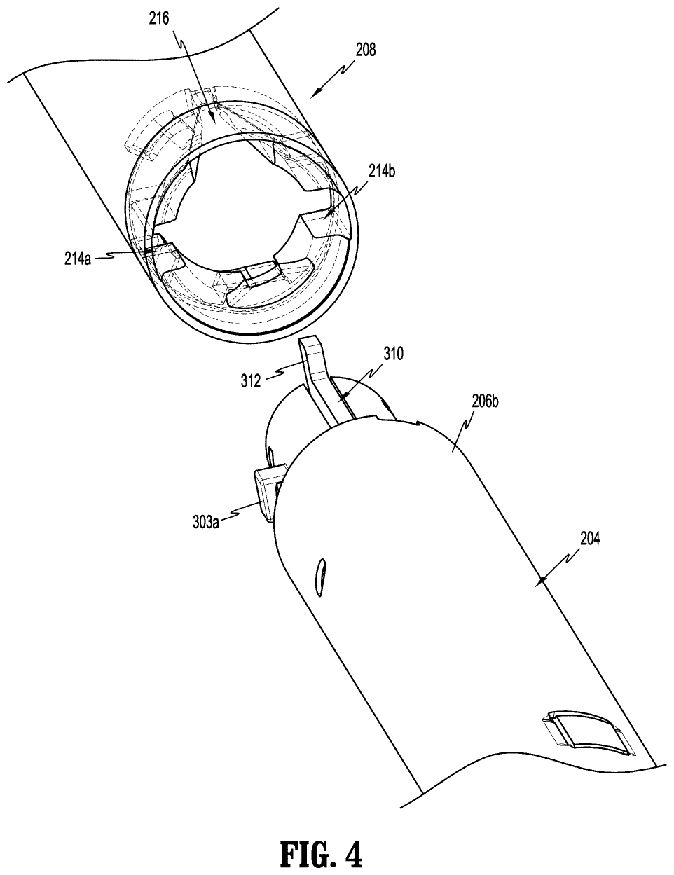

With reference to FIGS. 1, 3, and 4, the elongate body 204 of the adapter assembly 200 further includes a distal cap or ring member 208 extending distally from the distal end portion 206b. In aspects, the ring member 208 may be formed with the elongate body 204 and/or may be housed therein. The ring member 208 has an inner surface 210 that defines an opening or channel 212 configured for receipt of the proximal end portion 302a of the proximal body portion 302 of the surgical loading unit 300. The inner surface 210 of the ring member 208 further defines a pair of diametrically opposed apertures 214a, 214b and a slot 216 each being circumferentially disposed about the ring member 208. The slot 216 is disposed between the apertures 214a, 214b and is spaced circumferentially from each of the apertures 214a, 214b by about 90 degrees. The apertures 214a, 214b are configured for receipt of the respective pair of lugs 303a, 303b of the surgical loading unit 300 and the slot 216 is configured for receipt of the proximal end portion 310a (e.g., the flag 312) of the articulation link 310 of the surgical loading unit 300 during a proper insertion of the surgical loading unit 300 into the adapter assembly 200, as shown in FIG. 4.

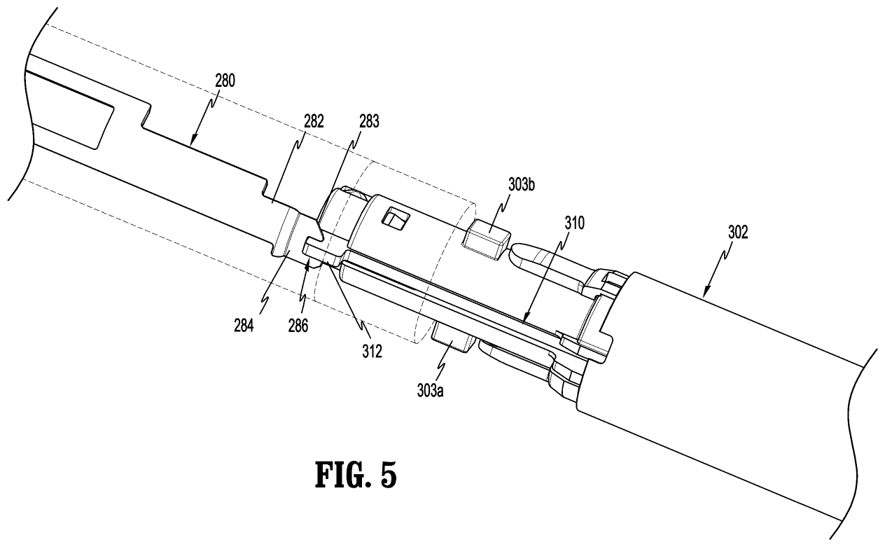

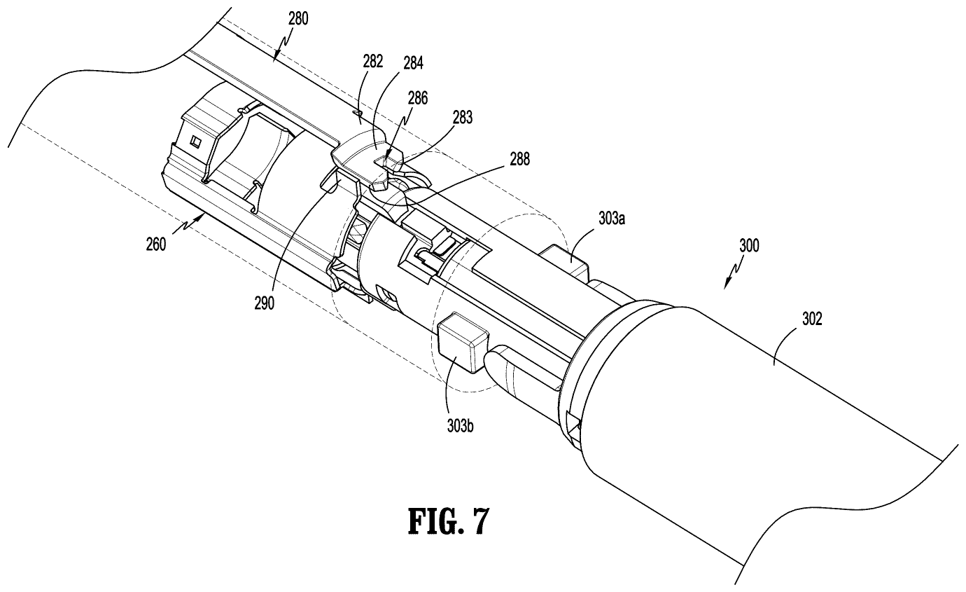

With reference to FIGS. 5 and 7, the adapter assembly 200 further includes an elongate loading bar or locking link 280 disposed within the elongate body 204 of the adapter assembly 200. The elongate loading bar 280 is slidingly disposed within the elongate body 204 and is resiliently biased toward a distal, locking position, as shown in FIG. 5. The elongate loading bar 280 has a distal extension 282 configured for locking engagement with the lug 303a (FIG. 2) of the surgical loading unit 300 upon the proper insertion of the surgical loading unit 300 into elongate body 204. The distal extension 282 has a distal end 284 having a distally-facing edge 283 defining a slot 286 therein. The slot 286 has a similar shape and size as the flag 312 of the surgical loading unit 300 to accommodate the flag 312 therein during an improper insertion of the surgical loading unit 300 into adapter assembly 200 (FIG. 5). The slot 286 as illustrated has a rectangular shape, but other suitable shapes are contemplated, such as rounded, triangular, or the like.

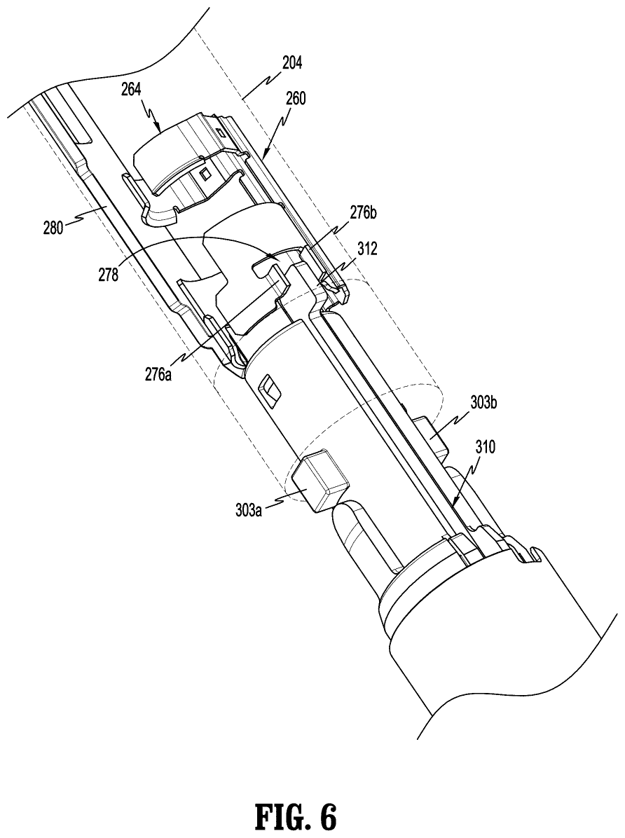

With reference to FIGS. 6 and 7, the adapter assembly 200 further includes an annular member 260 rotatably disposed within the elongate body 204 of the adapter assembly 200. The annular member 260 functions to electromechanically communicate to a processor (not shown) of the handle assembly 100 that the surgical loading unit 300 is either properly or improperly connected to the adapter assembly 200. In particular, upon rotating the annular member 260 relative to the elongate body 204, about a longitudinal axis of the elongate body 204, from a starting or first orientation to a second orientation, the annular member 260 transmits a signal to the processor of the handle assembly 100 indicating that the surgical loading unit 300 is secured to the adapter assembly 200 and is ready for use.

The annular member 260 defines a cylindrical passageway 264 therethrough configured for disposal of the proximal body portion 302 of the surgical loading unit 300. The annular member 260 includes a surface feature, such as, for example, a pair of tabs 276a, 276b defining a cavity 278 therebetween configured to interface with the lug 303b of the surgical loading unit 300, such that the annular member 260 is rotatable by and with the surgical loading unit 300 when the surgical loading unit 300 is properly inserted into the adapter assembly 200.

The annular member 260 further includes an appendage or additional surface feature 290 protruding radially outward therefrom and disposed on an opposite side of the annular member 260 as the pair of tabs 276a, 276b. The appendage or tab 290 is positioned in abutting engagement with a lateral edge surface 288 of the distal extension 282 of the elongate loading bar 280 (FIG. 7) when the elongate loading bar 280 is in the distal position. The elongate loading bar 280 prevents the annular member 260, and in turn, the surgical loading unit 300, from being rotated relative to the elongate body 204 due to the engagement of the appendage 290 of the annular member 260 with the elongate loading bar 280. As such, only when the lug 303a of the surgical loading unit 300 engages and proximally moves the elongate loading bar 280 out of engagement with the appendage 290 (during a proper insertion of the surgical loading unit 300) will the annular member 260 be able to be rotated by the surgical loading unit 300.

In operation, to properly assemble the surgical loading unit 300 with the adapter assembly 200, the surgical loading unit 300 is rotationally oriented (about a longitudinal axis thereof) so that the pair of lugs 303a, 303b of the surgical loading unit 300 are aligned with the pair of apertures 214a, 214b of the ring member 260 and the flag 312 of the articulation link 310 of the surgical loading unit 300 is aligned with the slot 216 of the ring member 208, as shown in FIG. 4. With the surgical loading unit 300 properly oriented, the surgical loading unit 300 may be translated toward the adapter assembly 200 to pass the proximal body portion 302 of the surgical loading unit 300 into the elongate body 204 of the adapter assembly 200 and, in turn, into the annular member 260. Upon fully inserting the surgical loading unit 300 into the adapter assembly 200, the lug 303b of the surgical loading unit 300 is received between the surface features 276a, 276b of the annular member 260, the lug 303a of the surgical loading unit 300 engages the elongate loading bar 280 to retract the elongate loading bar 280 towards its proximal position, and the flag 312 of the articulation link 310 couples to the articulation drive member (not shown) of the adapter assembly 200.

After moving the elongate loading bar 280 to the proximal position by the lug 303a of the surgical loading unit 300, the distal extension 282 of the elongate loading bar 280 is no longer engaged with the appendage 290 of the annular member 260, and therefore no longer preventing the annular member 260 from rotating out of the first orientation. With the surgical loading unit 300 in this initial insertion position within the adapter assembly 200, the surgical loading unit 300 is not yet lockingly engaged with the adapter assembly 200 and the annular member 260 remains in the first orientation. To complete the mechanical coupling of the surgical loading unit 300 to the adapter assembly 200, the surgical loading unit 300 is then rotated relative to the elongate body 204. Since the lug 303b of the surgical loading unit 300 is received in the cavity 278 defined between the surface features 276a, 276b of the annular member 260, rotation of the surgical loading unit 300 drives a rotation of the annular member 260 from the first orientation to the second orientation. Rotation of the annular member 260 from the first orientation to the second orientation establishes an electrical connection between the annular member 260 and the processor of the handle assembly 100, whereby the processor registers that the surgical loading unit 300 is lockingly engaged with the adapter assembly 200 and surgical instrument 10 is ready for operation.

For a more detailed description of the electrical connection between the annular member 260 and the processor, reference may be made to U.S. Pat. No. 10,314,579, the entire contents of which are incorporated by reference herein.

The rotation of the surgical loading unit 300 moves the lug 303a of the surgical loading unit 300 into an inner groove (not explicitly shown) defined in the ring member 208 of the elongate body 204 and out of a longitudinal path of the elongate loading bar 280. The resilient bias of the elongate loading bar 280 drives an axial translation thereof to dispose the elongate loading bar 280 in the distal or locking position. With the elongate loading bar 280 in the distal position, the lug 303a of the surgical loading unit 300 is captured between the ring member 208 and the distal extension 282, thereby preventing the surgical loading unit 300 from sliding or rotating out of the adapter assembly 200. In this state, the surgical loading unit 300 is properly releasably, lockingly engaged to the adapter assembly 200 and ready for use.

In some instances, it is possible for a clinician to inadvertently improperly orient the surgical loading unit 300 (about a longitudinal axis thereof) relative to the adapter assembly 200 prior to inserting the surgical loading unit 300 into the adapter assembly 200. For example, with reference to FIG. 5, the surgical loading unit 300 may be improperly oriented 90 degrees counter-clockwise (about the longitudinal axis thereof) from the proper orientation. When the rotational orientation of the surgical loading unit 300 is improper, the surgical loading unit 300 may still be longitudinally inserted into the adapter assembly 200. However, in this orientation, instead of the lug 303a of the surgical loading unit 300 engaging the distal extension 282 of the elongate loading bar 280, the flag 312 of the articulation link 310 is received in the slot 286 of the distal extension 282. Accordingly, when the clinician attempts to complete the assembly of the surgical loading unit 300 with the adapter assembly 200 by exerting a rotational force on the surgical loading unit 300, the engagement of the flag 312 of the articulation link 310 with the slot 286 of the elongate loading bar 280, which is non-rotatable, advantageously prevents the surgical loading unit 300 from being rotated. Therefore, the clinician will be unable to operate the surgical instrument 10 and will be alerted to the fact that the surgical loading unit 300 is improperly oriented.

With reference to FIGS. 6 and 7, the surgical loading unit 300 may be improperly oriented 90 degrees clockwise (about the longitudinal axis thereof) from the proper orientation. In this orientation, the flag 312 of the articulation link 310 is received in the cavity 278 defined by the pair of surface features 276a, 276b of the annular member 260 instead of the lug 303b of the surgical loading unit 300, as shown in FIG. 6. In addition, neither lug 303a nor lug 303b of the surgical loading unit 300 will engage the distal extension 282 of the elongate loading bar 280, such that the distal extension 282 remains engaged with the appendage 290 of the annular member 260. Accordingly, when the clinician attempts to complete the assembly of the surgical loading unit 300 with the adapter assembly 200 by exerting a rotational force on the surgical loading unit 300, the engagement of the appendage 290 with the distal extension 282 advantageously prevents the annular member 260, and in turn, the surgical loading unit 300 from being rotated. Therefore, the clinician will be unable to operate the surgical instrument 10 and will be alerted to the fact that the surgical loading unit 300 is improperly oriented.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1-20. (canceled)

21. A surgical instrument, comprising:
a surgical loading unit, comprising:
a body defining a longitudinal axis and comprising at least one lug projecting from the body; and
a tool assembly coupled to a distal end portion of the body; and
an adapter assembly, comprising:
an elongate body having a proximal end and a distal end;
a loading bar located within the elongate body and comprising a distal extension with a first slot; and
a ring member disposed at the distal end of the elongate body and configured to receive the surgical loading unit when inserted in a first rotational position and in a second rotational position, the ring member comprising at least one aperture configured to receive the at least one lug of the surgical loading unit when inserted in the first rotational position and in the second rotational position;
wherein when the surgical loading unit is inserted in the first rotational position, the first slot of the distal extension is configured to lockingly engage the surgical loading unit for preventing rotation toward an assembled position within the elongate body.

22. The surgical instrument of claim 21, wherein the surgical loading unit comprises an articulation link receivable by the first slot for locking engagement when inserted in the first rotational position.

23. The surgical instrument of claim 22, wherein the ring member further comprises a second slot configured to receive the articulation link for engaging a drive assembly when inserted in the second rotational position.

24. The surgical instrument of claim 23, wherein the articulation link comprises a flag configured to align with the second slot of the ring member for coupling to a drive assembly when in the assembled rotational position.

25. The surgical instrument of claim 24, wherein the flag is receivable within the first slot of the loading bar for locking engagement when inserted in the first rotational position.

26. The surgical instrument of claim 23, wherein the at least one aperture comprises a first aperture and a second aperture, and the at least one lug comprises a first lug and a second lug receivable within the respective first aperture and the second aperture.

27. The surgical instrument of claim 26, wherein when the surgical loading unit is inserted in the second rotational position, the first lug is configured to abut the distal extension for retracting the loading bar to a proximal position without locking engagement with the first slot.

28. The surgical instrument of claim 27, wherein when the loading bar is in the proximal position, the surgical loading unit is rotatable within the elongate body from the second rotational position toward the assembled rotational position.

29. The surgical instrument of claim 28, wherein when the surgical loading unit is in the assembled rotational position, the second lug is secured between the ring member and the distal extension.

30. The surgical instrument of claim 26, wherein the first aperture and the second aperture are disposed on opposing sides of the ring member.

31. The surgical instrument of claim 25, wherein the first slot is spaced circumferentially from the second slot by 90 degrees such that the first rotational position is spaced from the second rotational position by 90 degrees.

32. An adapter assembly for a surgical loading unit, comprising:
an elongate body having a proximal end and a distal end;
a loading bar located within the elongate body and comprising a distal extension with a first slot; and
a ring member disposed at the distal end of the elongate body and configured to receive the surgical loading unit when inserted in a first rotational position and in a second rotational position, the ring member comprising at least one aperture configured to receive a corresponding at least one lug of the surgical loading unit when inserted in the first rotational position and in the second rotational position;
wherein when the surgical loading unit is inserted in the first rotational position, the first slot of the distal extension is configured to lockingly engage the surgical loading unit for preventing rotation toward an assembled position within the elongate body.

33. The adapter assembly of claim 32, wherein the surgical loading unit comprises an articulation link receivable by the first slot for locking engagement when inserted in the first rotational position.

34. The adapter assembly of claim 33, wherein the ring member further comprises a second slot configured to receive the articulation link for engaging a drive assembly when inserted in the second rotational position.

35. The adapter assembly of claim 34, wherein the articulation link comprises a flag configured to align with the second slot of the ring member for coupling to a drive assembly when in the assembled rotational position.

36. The adapter assembly of claim 35, wherein the flag is receivable within the first slot of the loading bar for locking engagement when inserted in the first rotational position.

37. The adapter assembly of claim 34, wherein the at least one aperture comprises a first aperture and a second aperture.

38. The adapter assembly of claim 37, wherein when the surgical loading unit is inserted in the second rotational position, the distal extension is arranged to abut the at least one lug for retracting the loading bar to a proximal position without locking engagement with the first slot.

39. The adapter assembly of claim 38, wherein the first aperture and the second aperture are disposed on opposing sides of the ring member.

40. The adapter assembly of claim 39, wherein the first slot is spaced circumferentially from the second slot by 90 degrees such that the first rotational position is spaced from the second rotational position by 90 degrees.
